(19) United States
(12) Patent Application Publication
(10) Pub. No.: US 2018/0235521 A1
(43) Pub. Date: Aug. 23, 2018

(54) HEXAGONAL NANOFLUIDIC MICROCHANNELS FOR BIOFLUID SENSING DEVICES

(71) Applicant: Eccrine Systems, Inc., Cincinnati, OH (US)

(72) Inventor: Jason Heikenfeld, Cincinnati, OH (US)

(73) Assignee: Eccrine Systems, Inc., Cincinnati, OH (US)

(21) Appl. No.: 15/958,725

(22) Filed: Apr. 20, 2018

Related U.S. Application Data
(63) Continuation-in-part of application No. 15/746,452, filed on Jan. 22, 2018.
(60) Provisional application No. 62/633,210, filed on Feb. 21, 2018.
(60) Provisional application No. 62/196,541, filed on Jul. 24, 2015.
(60) Provisional application No. 62/208,171, filed on Aug. 21, 2015.

(51) Int. Cl. A61B 5/145; A61B 5/00; B01L 3/00

(52) U.S. Cl. CPC A61B 5/14517; A61B 5/6801; B01L 3/502707; B01L 3/502715; B81B 7/0006; B01L 2300/0627; B01L 2300/0864; B01L 2300/161; B01L 2400/04; B01L 3/50273

(57) ABSTRACT

The disclosed invention provides a biofluid collection device configured with a hexagonal open microfluidic network, which facilitates nanoliter-scale biofluid collection and transport for biosensing applications. In one embodiment, a biofluid sensing device placed on the skin for measuring a characteristic of an analyte in sweat includes one or more biofluid sensors and a hexagonal open microfluidic network biofluid collector. The disclosed collector provides a volume-reduced pathway for sweat biofluid between the one or more sensors and sweat glands when the device is positioned on the skin. In another embodiment, a biofluid collector includes a network of microchannels comprising three or more repeatedly intersecting channels that provide redundant pathways for biofluid transport.

### CROSS-REFERENCE TO RELATED APPLICATIONS

This application is a continuation-in-part of U.S. application Ser. No. 15/746,452, filed Jan. 22, 2018; and claims priority to U.S. Provisional Application No. 62/633,210, filed Feb. 21, 2018; as well as PCT/US16/43771, filed Jul. 23, 2016, the disclosures of which are hereby incorporated by reference herein in their entirety.

### BACKGROUND OF THE INVENTION

This application has specification that builds upon Twine, N., et al., “Open Nanofluidic Films with Rapid Transport and No Analyte Loss for Ultra-Low Sample Volumes,” Lab on a Chip, 2018, which is hereby incorporated by reference herein in its entirety.

Sweat contains many of the same biomarkers, chemicals, or solutes that are carried in blood and can provide significant information enabling one to diagnose illness, health status, exposure to toxins, performance, and other physiological attributes even in advance of any physical sign. Furthermore, sweat itself, the action of sweating, and other parameters, attributes, solutes, or features on, near, or beneath the skin can be measured to further reveal physiological information. Of the other physiological fluids used for biological monitoring (e.g., blood, urine, saliva, tears), sweat has arguably the least predictable sampling rate in the absence of technology. However, with proper application of technology, sweat can be made to outperform other non-invasive or less invasive biofluids in predictable sampling.

However, the state of art in sweat bio monitoring is in need of additional devices and methods to properly reduce the dead volume between sensors and skin. Reducing dead volume reduces the amount of sweat required to reliably transport a sweat sample across sensors, and reduces the opportunity for newer sweat to mix with older sweat, which mixing confounds chronological measurements. Further, transporting a very low volume of sweat to sensors is critical to achieve fast sampling times, or for sampling during intervals with very low sweat rates. In addition, it also may be critical for prolonged stimulation (i.e., in order to minimize stimulation), and for improving biomarker measurements where a low sweat rate is required to ensure correlation between biomarker concentrations in sweat and those in blood.

While techniques for transporting microliter sample volumes to sensors for analyte sensing is now technologically mature, current solutions in the art are often ill-suited to applications in the nanoliter regime (<100 nL). Challenges associated with nanoliter transport to sensors as well as interface with sensors include difficulties in sensor integration with the transport means, increased resistance to fluid flow, and prohibitive amounts of analyte exchange between the sample and the transport medium. For example, in sweat sensing applications recent work to reduce sample volumes by using an ˜8 μL microchannel and a sweat collection area of ˜0.1 cm² still requires an 8.5-hour collection time at conventional sweat generation rates (-1 nL/min/gland). Similarly, existing wicking materials have shown inadequacy for sweat sensing applications due to excessive analyte exchange. For example, Rayon™ has advantageous properties for reducing sample volume, since its structure allows fluid transport along wicking nano-grooves, without the need to wet the entire material. However, analyte exchange with Rayon fabric is so prevalent that even high concentration analytes such as electrolytes (10's mM), can become sufficiently depleted in the sweat sample to prevent rapid sensing of concentration changes. Other widely used wicking materials are even more problematic for low concentration analytes, e.g., PDMS readily adsorbs hydrophobic small molecules, such as hormones, that are found in nM unbound concentrations in sweat.

Therefore, what is needed are materials and methods to provide biofluid transport and sensor interface at the nanoliter scale that allow for responsive and continuous sensing of low concentration analytes.

### SUMMARY OF THE INVENTION

The disclosed invention provides a biofluid collection device configured with a hexagonal open microfluidic network, which facilitates nanoliter-scale biofluid collection and transport for biosensing applications. In one embodiment, a biofluid sensing device placed on the skin for measuring a characteristic of an analyte in sweat includes one or more biofluid sensors and a hexagonal open microfluidic network biofluid collector. The disclosed collector provides a volume-reduced pathway for sweat biofluid between the one or more sensors and sweat glands when the device is positioned on the skin. In another embodiment, a biofluid collector includes a network of microchannels comprising three or more repeatedly intersecting channels that provide redundant pathways for biofluid transport.

### BRIEF DESCRIPTION OF THE DRAWINGS

The disclosed invention will be further appreciated in light of the following descriptions and drawings in which:

FIG. 1 depicts at least a portion of a device comprising the disclosed invention.

FIG. 2 depicts at least a portion of a device comprising an open nanofluidic film for low volume biofluid transport.

FIG. 3 depicts at least a portion of a device comprising an open nanofluidic film for low volume biofluid transport.

FIG. 4 depicts at least a portion of a device comprising an open nanofluidic film for low volume biofluid transport.

FIG. 5 depicts at least a portion of a device comprising an open nanofluidic film for low volume biofluid transport.

FIG. 6 depicts at least a portion of a microchannel cross section of the disclosed invention.

FIG. 7 depicts at least a portion of a microchannel cross section of the disclosed invention.

### DEFINITIONS

“Chronological assurance” means a sampling rate or sampling interval for measurement(s) of sweat, or solutes in sweat, at which measurements can be made of new sweat or its new solutes as they originate from the body. Chronological assurance may also include a determination of the effect of sensor function, or potential contamination with previously generated sweat, previously generated solutes, other fluid, or other measurement contamination sources for the measurement(s).

“Sweat sampling rate” means the effective rate at which new sweat, or sweat solutes, originating from the sweat gland or from skin or tissue, reaches a sensor that measures a property of sweat or its solutes. Sweat sampling rate, in some cases, can be far more complex than just sweat generation rate. Sweat sampling rate directly determines, or is a contributing factor in determining chronological assurance. Times and rates are inversely proportional (rates having at least partial units of 1/seconds), therefore a short or small time required to refill a sweat volume can also be said to have a fast or high sweat sampling rate.

The inverse of sweat sampling rate (1/s) could also be interpreted as a “sweat sampling interval”. Sweat sampling rates or intervals are not necessarily regular, discrete, periodic, discontinuous, or subject to other limitations. Like chronological assurance, sweat sampling rate may also include a determination of the effect of potential contamination with previously generated sweat, previously generated solutes, other fluid, or other measurement contamination sources for the measurement(s). Sweat sampling rate can also be in whole or in part determined from solute generation, transport, advective transport of fluid, diffusion transport of solutes, or other factors that will impact the rate at which new sweat or sweat solutes reach a sensor and/or are altered by older sweat or solutes or other contamination sources. Sensor response times may also affect sampling rate.

“Sweat generation rate” means the rate at which sweat is generated by the sweat glands themselves. Sweat generation rate is typically measured by the flow rate from each gland in nL/min/gland. In some cases, the measurement is then multiplied by the number of sweat glands from which the sweat is being sampled.

“Measured” may mean an exact or precise quantitative measurement and can include broader meanings such as, for example, measuring a relative amount of change of something. Measured can also mean a binary measurement, such as ‘yes’ or ‘no’ type measurements.

“Sweat volume” means the fluidic volume in a space that can be defined multiple ways. Sweat volume may be the volume that exists between a sensor and the point of generation of sweat, or between a sensor and a solute moving into or out of sweat from the body or from other sources. Sweat volume can include the volume that can be occupied by sweat between the sampling site on the skin and a sensor on the skin, where the sensor has no intervening layers, materials, or components between it and the skin; or between the sampling site on the skin and a sensor on the skin where there are one or more layers, materials, or components between the sensor and the sampling site on the skin. Sweat volume may refer to the sweat volume of multiple integrated components, or used in description of the sweat volume for single component or a subcomponent, or in the space between a device, or device component, and skin.

“Volume-reducing component” means any component, material, element, or feature of the present disclosure that facilitates the creation of a volume-reduced pathway.

“Volume-reduced pathway” means a sweat volume that has been reduced by the addition of a material, device, layer, or other component, which therefore decreases the sweat sampling interval for a given sweat generation rate. Specific to the instant disclosure, a volume reduced pathway refers to any combination of elements disclosed herein that at least in part uses wicking pressure to enable the formation of the volume reduced pathway. For example, a volume reduced pathway could be created in the space between a sweat collector and skin by wicking sweat through this space. The disclosed invention may benefit from additional methods to reduce the sweat volume, but if the term volume-reduced pathway is used herein, then wicking pressure must, at least in part, enable or create the volume-reduced pathway.

“Microfluidic components” means channels in polymer, textiles, paper, or other components known in the art of microfluidics for guiding movement of a fluid or at least partial containment of a fluid.

“Nanofluidic wicking” means channels that transport biofluids on a nanoliter (10⁻⁹ L) scale.

“Peptide” means short chains of amino acid monomers, i.e., less than around 50 amino acid monomers, linked by amide bonds.

### DETAILED DESCRIPTION OF THE INVENTION

The disclosed invention includes a design for a hexagonal wick (“hex wick”) which addresses major challenges in nanoscale biofluid transport and sensing through the incorporation of several innovative features: (1) the wick achieves an effective wicking film thickness of ˜1 μm (<100 nL/cm²) through a hexagonal network of ˜10×10 μm open channels that comprise ˜10% of the open surface area; (2) analyte exchange with the wick is substantially prevented by use of a thin gold coating; (3) rapid wicking transport through rectangular microchannels reduces resistance to fluid flow as compared to traditional wicking materials; (4) ease of manufacture; (5) hydrophilicity provided through a shelf-stable and biologically safe peptide surface modification; (6) hydrophilicity allows omnidirectional wicking beyond corner junctions as compared to traditional linear wicking; (7) specific to sweat biosensing, the wick also reduces the dead volume against the skin surface which reduces contamination from the stratum corneum.

To clarify the proper numerical values or representations of sweat sampling rate and therefore chronological assurance, sweat generation rate and sweat volumes will be described in detail. From Dermatology: an illustrated color text, 5th ed., the maximum sweat generated per person per day is 10 L, which on average is 4 μL per gland maximum per day, or about 3 nL/min/gland. This is about 20× higher than the minimum sweat generation rate. The maximum stimulated sweat generation rate according to Buono 1992, J. Derm. Sci. 4, 33-37, “Cholinergic sensitivity of the eccrine sweat gland in trained and untrained men,” the maximum sweat generation rate by pilocarpine stimulation is about 4 nL/min/gland for untrained men and 8 nL/min/gland for trained (exercising often) men. Sweat stimulation data from “Pharmacologic responsiveness of isolated single eccrine sweat glands,” by K. Sato and F. Sato, Am. Physiological Society, Jul. 30, 1980, suggests a sweat generation rate up to about 5 nL/min/gland is possible with stimulation, and several types of sweat stimulating substances are disclosed (the data was for extracted and isolated monkey sweat glands, which are very similar to human ones). For simplicity, we can assume for calculations in the present disclosure (without so limiting the disclosure), that the minimum sweat generation rate is about 0.1 nL/min/gland, and the maximum sweat generation rate is about 5 nL/min/gland, which is about a 50× difference between the maximum and minimum rates.

Based on the assumption of a sweat gland density of 100/cm², a sensor that is 0.55 cm in radius (1.1 cm in diameter) would cover about 1 cm² area, or approximately 100 sweat glands. Next, assume a sweat volume under a skin-facing sensor (space between the sensor and the skin) of 100 μm average height or 100E-4 cm, and that same 1 cm² area, which provides a sweat volume of 100E-4 cm³ or about 100E-4 mL or 10 μL of volume. With the maximum sweat generation rate of 5 nL/min/gland and 100 glands, it would require 20 minutes to fully refresh the sweat volume (using first principles/simplest calculation only). With the minimum sweat generation rate of 0.1 nL/min/gland and 100 glands, it would require 1000 minutes or ∥17 hours to refresh the sweat volume. Because the flow is not entirely centered, according to Sonner, et al., in Biomicrofluidics, May 15, 2015; 9(3):031301. doi: 10.1063/1.4921039, the time to fully refresh the sweat volume (i.e., new sweat replaces all old sweat) could be six times longer or more. For slow sweat flow rates, back-diffusion of analytes and other confounding factors could make the effective sampling interval even larger. Clearly, conventional wearable sweat sensing approaches with large sweat volumes and slow sampling rates would find continuous sweat sample monitoring to be a significant challenge.

Sweat stimulation, or sweat activation, can be achieved by known methods. For example, sweat stimulation can be achieved by simple thermal stimulation, chemical heating pad, infrared light, by orally administering a drug, by intradermal injection of drugs such as carbachol, methylcholine or pilocarpine, and by dermal introduction of such drugs using iontophoresis, by sudo-motor-axon reflex sweating, or by other means. A device for iontophoresis may, for example, provide direct current and use large lead electrodes lined with porous material, where the positive pole is dampened with 2% pilocarpine hydrochloride or carbachol and the negative one with 0.9% NaCl solution. Sweat can also be controlled or created by asking the device wearer to conduct or increase activities or conditions that cause them to sweat.

The present disclosure applies at least to any type of sweat sensing device that stimulates sweat, measures sweat, sweat generation rate, sweat chronological assurance, its solutes, solutes that transfer into sweat from skin, a property of or things on the surface of skin, or properties or things beneath the skin. The disclosed invention, in all embodiments, includes at least one sensor that is specific to an analyte in sweat. To clarify further, just measuring sweat conductivity is not specific to one analyte because it measures the sum of conductance contributed by all ionic solutes in sweat. However, an ion-selective electrode configured to detect potassium is a sensor specific to one analyte. As an additional example, a sensor for sweat cortisol that only has interference (non-specificity) to estrogen, would still be specific to one analyte as described herein, since there are many device applications in which estrogen concentrations are static, but cortisol concentrations would change, making the sensor effectively specific to cortisol. Any suitable sensor may be used in the disclosed invention (e.g., ion-selective, enzymatic, antibody, aptamer, optical, electrical, mechanical, etc.). The disclosure applies to sweat sensing devices with various configurations including patches, bands, straps, portions of clothing, wearables, or any suitable mechanism that reliably brings sweat stimulating, sweat collecting, and/or sweat sensing technology into intimate proximity with sweat as it is generated. Some embodiments use adhesives to hold the device near the skin, but devices may also be secured by another suitable mechanism, such as a strap or helmet suspension.

Certain embodiments of the disclosure describe sensors as simple individual elements. It is understood that many sensors require two or more electrodes, reference electrodes, or additional supporting technology or features that are not captured in the description herein. Sensors are preferably electrical in nature, but may also include optical, chemical, mechanical, or other known biosensing mechanisms. Sensors can be in duplicate, triplicate, or more, to provide improved data and readings. Sensors may be referred to by what the sensor is sensing, for example: a sweat sensor; an impedance sensor; a sweat volume sensor; a sweat generation rate sensor; or a solute generation rate sensor. Certain embodiments of the disclosed invention show sub-components that may require additional obvious sub-components for use of the device in various applications (such as a battery), and for purpose of brevity and focus on inventive aspects are not explicitly shown in the diagrams or described in the embodiments of the present disclosure. As a further example, many embodiments of the disclosed invention may benefit from mechanical or other means to keep the devices or sub-components firmly affixed to skin or to provide pressure facilitating constant contact with skin or conformal contact with ridges or grooves in skin, as are known to those skilled in the art of wearable devices, patches, bandages, or other technologies or materials that are affixed to skin. Such means are included within the spirit of the disclosed invention. The present application has specification that builds upon PCT/US13/35092, the disclosure of which is hereby incorporated herein by reference in its entirety.

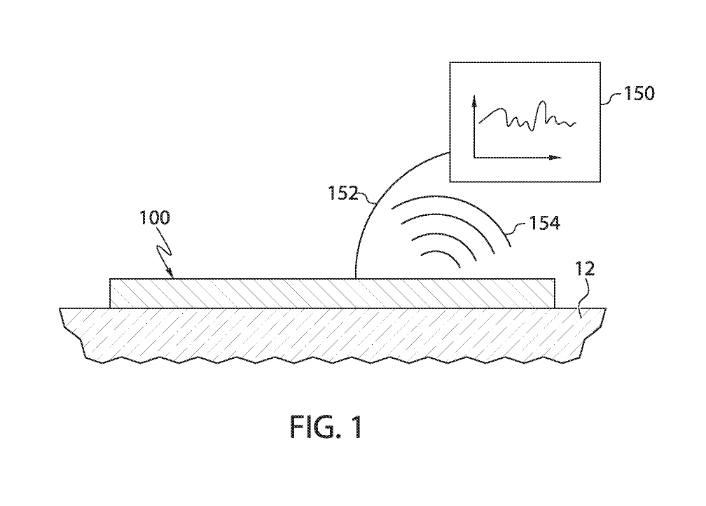

With reference to FIG. 1, a sweat sensing device 100 is placed on or near skin 12. In an alternate embodiment, the sweat sensing device may be simply fluidically connected to skin or regions near skin through microfluidics or other suitable techniques. The device 100 is in wired communication 152 or wireless communication 154 with a reader device 150. In some embodiments, reader device 150 may be a smart phone or portable electronic device. In alternate embodiments, device 100 and reader device 150 can be combined. In further alternate embodiments, communication 152 or 154 is not constant and could be a one-time data transmission from device 100 once it has completed its measurements of sweat.

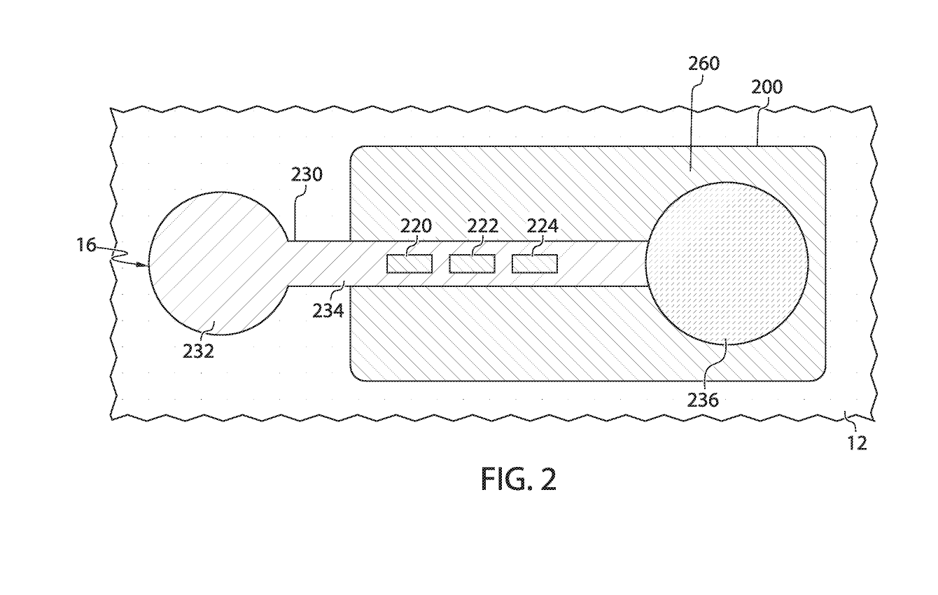

FIG. 2 depicts an overhead view of a wearable biofluid sensing device 200 as it is worn on skin 12. The device includes a fluid impermeable substrate 260, made from, e.g., PET, PVC; and a microfluidic wick 230, which includes a wicking collector 232 and a wicking coupler 234. The wicking coupler 234 may be constructed of a polymer, paper, textile, rayon, or other suitable material for transporting the biofluid sample across one or more biofluid sensors 220, 222, 224 and facilitating the interface between the sweat sample and sensors. The microfluidic wick 230 is in fluidic communication with the skin 12, a wicking pump 236, and the one or more sensors 220, 222, 224.

The wicking pump 236 is constructed of paper, or may be an absorbent hydrogel, a desiccant, or other material suitable for drawing a biofluid sample across and away from the sensors. The wicking pump 236 should have sufficient volume to sustain operation of the device throughout the application's intended duration (i.e., it should not become saturated during device operation). For example, if the device is to be used for 24 hours, then neither microfluidic wick 230 nor the wicking pump 236 should become fully saturated with sweat during the 24 hours of operation. In some embodiments, microfluidic wick 230 and wicking pump 236 may be the same material or component.

The sensors include one or more analyte specific sensors 220, 222, e.g., ion-selective electrode sensors, electrochemical aptamer-based sensors, amperometric, or enzymatic sensors. Some embodiments also include one or more secondary sensors 224, which may be, e.g., volumetric sweat rate, micro-thermal flow rate, GSR, sweat conductivity, impedance or capacitance sensors for skin contact measurement, or a temperature sensor.

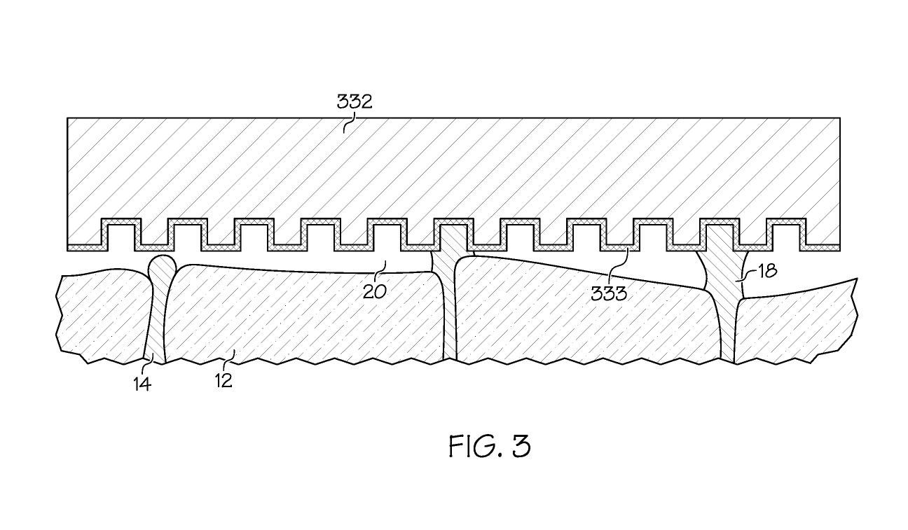

With reference to FIG. 3, which depicts the wicking collector 232 of FIG. 2 as viewed from the direction of the arrow 16, the wicking collector 332 interacts with the skin 12 of the device wearer. The wicking collector 332 is comprised of a polymer having a skin-facing surface that contains a plurality of interconnected microchannels 333 arranged in a hexagonal pattern. The microchannels have dimensions of, e.g., 10 μm width and 15 μm height, and preferably have substantially square (not rounded) corners. The channels may be manufactured in a variety of ways, such as laser etching the channels into the polymer. Other techniques include casting the channels by pouring the polymer into a mold bearing the desired pattern, and then curing the polymer. The wicking collector 332 may be constructed of any material that allows gold adhesion and can achieve the required geometric shape. Alternatively, the wicking collector 332 may be constructed of a simple hydrophilic polymer, or a polymer, e.g., PET, that is treated or coated to be hydrophilic or super-hydrophilic, such as by coating with a nano-silica, or a hydrogel such as agar. Between the skin 12 and the wicking collector 332, is a wicking space or dead volume 20. As sweat 14 leaves the skin, it first forms droplets, and when sufficient sweat is produced by the sweat gland, it wets 18 the wicking collector 332, and enters the microchannels 333, where it is transported to the wicking coupler (not shown).

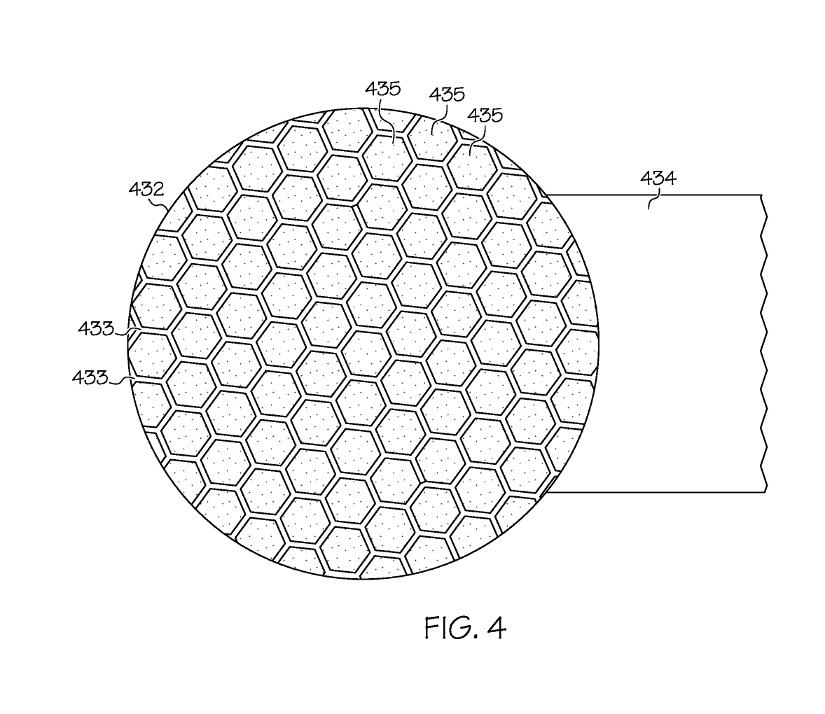

With reference to FIG. 4, the underside of wicking collector 232 of FIG. 2 is depicted. The skin-facing side of the wicking collector 432, comprises a plurality of open interconnecting microchannels 433 that create a plurality of hexagonal structures 435 between the channels. The hexagonal structures 435 and microchannels 433 have a hydrophilic gold coating, e.g., a sputter-deposited 10 nm gold coating, to reduce contamination from skin. The microchannels create a hexagonal network of open surface channels and intervening hexagonal structures, a hex wick, which satisfies a number of requirements for nanoscale biofluid transport, that include: transport of ultra-low biofluid volumes; minimized surface-area to volume; no or negligible analyte exchange with the hex wick, and simplicity of manufacture. Regarding simplicity of manufacture, large sheets of hex wicks can be fabricated, and then cut to size and laminated against other components, such as the wicking coupler 434, sensors (not shown), or additional hex wicks (not shown) to construct a biofluid sensing device.

The hex wick as disclosed also provides a number of advantages over other biofluid collection configurations. For example, compared to a sweat collector with a single continuous channel, the hex wick provides multiple redundant paths for a biofluid sample to reach the sensors. If the single channel were to suffer a blockage, break, or other defect, the wicking and sweat transport capability of the entire wicking collector could be disrupted. A hex wick, however, provides redundancy in potential wicking paths, meaning that a broken sub-channel will not prevent the network from wicking and transporting sweat. Therefore, embodiments of the disclosed invention may include a network of at least partially redundant wicking pathways.

Another advantage of the disclosed hex wick is the ability to provide greater contact area between wicking channels and sweat gland openings relative to existing biofluid collector materials. For example, a simple textile sweat collector with random fiber arrangement (e.g., non-woven) could have areas with poor local contact to skin, and therefore in some areas would require more sweat volume in order to allow wicking connection between the opening of a sweat gland on the skin surface and the textile. The disclosed hex wick, however, can be precisely configured so that there is no more than 500 μm, and preferably no more than 100 μm, distance between adjacent wicking pathways in the hex wick, thereby providing consistently small distances between wicking pathways and sweat glands, and in turn an overall reduction in sweat volume required by the device.

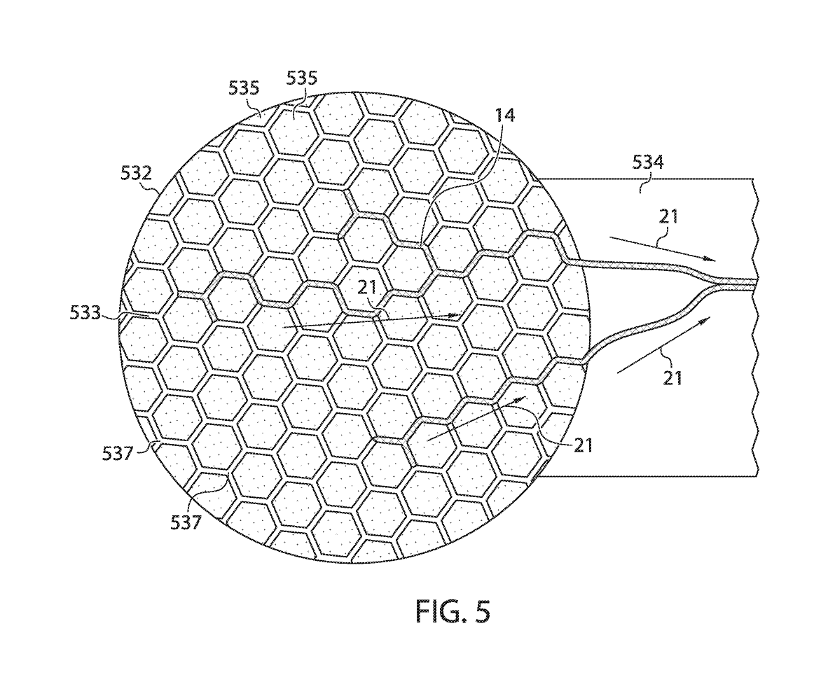

With reference to FIG. 5, the underside of the wicking collector 432 of FIG. 4 is depicted under active sweating conditions. As sweat 14 wets into the microchannels 533, it wicks along the channel pathways in the direction of the arrows 21 to the wicking coupler 534, and to the sensors (not shown). The mechanics of fluid transport in the hex wick are quite sophisticated, particularly due to the divergent capillary dimensions of the microchannels 533 that exist at the connecting junctions 537. Several wicking principles are required to characterize the fluid flow through the hex wick, and will be described here in the order of difficulty for achieving continuous wicking through the microchannels. The easiest model available is capillary flow through microchannels, wherein the channels are modeled as open u-channels with perfectly square corners. However, due to manufacturing difficulty, the microchannels will have somewhat rounded corners. As a result, more complex models will have to be used, including modeling capillary flow through open u-channels with rounded corners, and modeling the flow of capillary filaments propagating along open u-channels with rounded corners.

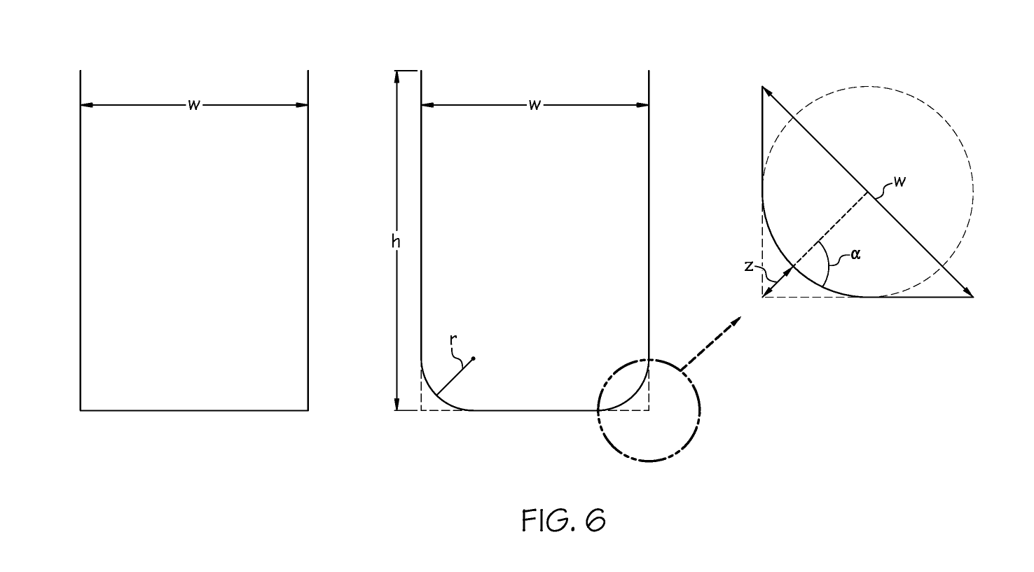

With reference to FIG. 6, the simplest model of capillary flow through a hex wick microchannel is to treat the open u-channel as a combination of two perfectly square corners. The u-channel corner wicking is determined by the channel aspect ratio: width (w) and height (h), and Young's contact angle (θ). For an open u-channel with perfectly square corners, the condition for capillary flow is:

$$\frac{w}{2 h + w} < cos θ .$$

Thus, for an aspect ratio of 1.5 (10 μm width and 15 μm height), the contact angle necessary to satisfy capillary flow is <75°. Maintaining such a low contact angle is trivial, but real-world fabrication methods will likely have corner rounding with a radius (r), resulting in a more challenging condition for capillary flow:

$$\frac{w}{2 h + w} + \frac{wr ( 4 - π )}{2 h + {w}^{2}} < cos θ .$$

Using this equation, even where corner rounding is worst-case, i.e., the corner radius is equal to the 10 μm width of the channel, the contact angle necessary for capillary flow is 66°, which is also trivial to achieve with many hydrophilic materials.

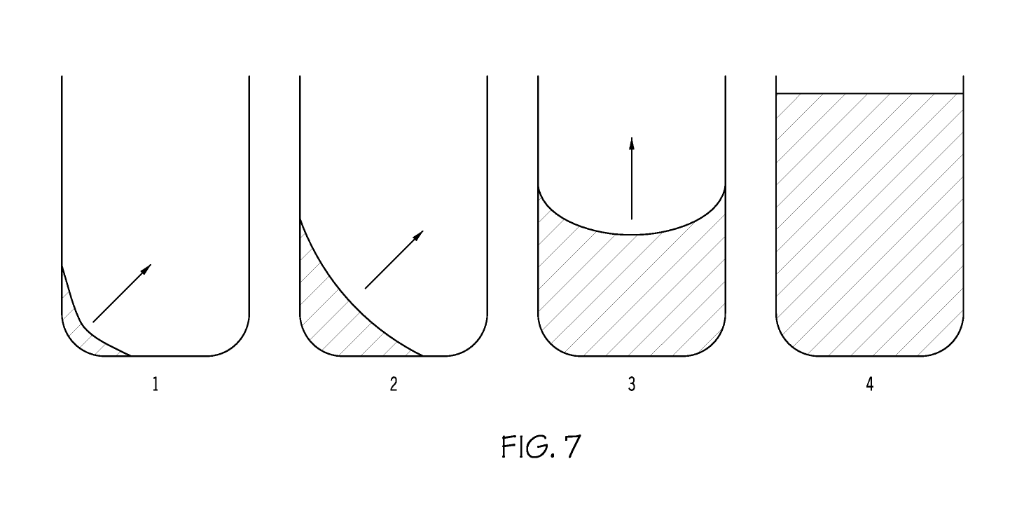

However, because the hex wick has divergent capillary geometries at the channel junctions, a third more difficult requirement exists: unless more difficult-to-make high-aspect-ratio channels are utilized, capillary filaments along the corners are necessary to promote continuous wicking. The requirement for capillary filaments is best understood by examining how fluid wets and fills the microchannel. As fluid enters a u-channel, it propagates in a repeating pattern comprising four main steps, as depicted in FIG. 7. 1) a capillary filament occurs at the corners of the channels, and travels ahead of the bulk capillary flow; 2) the capillary filament has a concave meniscus and therefore due to Laplace pressure also fills the corner into the cross-section of the channel; 3) the capillary filament reaches the other channel side wall, and a new concave meniscus is formed which then further fills the channel due to Laplace pressure; 4) the filled channel then supports bulk capillary flow, which follows additional capillary filaments traveling ahead of the bulk flow. In the hex wicks disclosed herein, the capillary filaments propagate so quickly that they surround an entire hexagon perimeter before channel filling occurs. It should be noted that although the maximum volume of the hex wick is ˜150 nL/cm² (10×15 μm channels, 10% of surface area), during use with a hydrogel or cellulose wicking pump it is unlikely the channels will be fully filled, and the volume during use is likely <100 nL/cm².

Because the hex wick requires the described capillary filaments to promote continuous wicking, choice of materials becomes a major challenge. A capillary filament can be understood by representing the corners of the channels as rounded v-grooves with dimensions discussed for previous examples, and can be modeled as:

$$sin α ( 1 + 2 α \frac{z}{w} ) < cos θ .$$

Using the same numbers described previously, and assuming a corner rounding radius of 1 μm, the necessary contact angle is <35°. Achieving this contact angle will require coating the microchannels with a functionalization coating to promote capillary filament propagation. Such a functionalization coating must meet certain criteria, namely, it first must be compatible with the gold coating which covers the hex wick polymer. Second, the functionalization coating must be biologically compatible, and should be generally regarded as safe (GRAS) for skin contact during biofluid sensing applications, even if the functionalization coating becomes detached from the hex wick. Examples of thiols that would be suitable for such a purpose include monothiol thioglycolic acid (TGA), sodium 3-mercapto-1-propanesulfonate (MPS), both of which showed the required contact angle of <30°. Other materials showing better long-term stability include peptides, e.g., 5mer (2 cysteine groups, dithiol) and 7mer (3 cysteine groups, trithiol) peptides, with aspartic acid as the additional group to improve hydrophilicity.

Under in vivo test conditions, the invention as disclosed achieved electrode response within 3 minutes after the initiation of sweat stimulation. This timing is the fastest sweat-to-sensor transport time currently known in the art, and roughly agrees with the modeled transport times. For example, a hex wick used as described has 10×15 μm channels at 10% of the area, and therefore ˜150 nL/cm² maximum volume. If the sweat generation rate is approximately 500 nL/min/cm² (as measured with a gravimetric sweat collector), then 1 cm² of the wick should fill up in 18 seconds (hex wick volume/sweat generation). The actual collection area used of 0.95 cm² should also provide an input sweat flow rate of 475 nL/min. Next, the maximum volume of the remainder of the hex wick is 60 nL, and the volume of the wicking coupler on the electrodes is ˜6% of total volume, ˜270 nL. The total volume is therefore 480 nL and the sensors should all respond within 500 nL/475 nL/minute, or approximately 60 seconds.

The above-described configurations represent a basic foundation for either a simple device or a more complex device. Some embodiments of the disclosed invention may therefore include additional materials, components, designs, or other features for operation, as long as the device uses at least one wicking component, or operates at least in part by wicking pressure. More generally, regardless of how a wicking collector, a wicking pump, or a wicking coupler are configured, arranged, or omitted from a device of the present disclosure, the wicking pressure(s) are such that the sensor(s) is able to receive adequate sweat to perform accurate measurements during device operation.

This has been a description of the present invention along with a preferred method of practicing the present invention, however the invention itself should only be defined by the appended claims.

What is claimed is:

1. A biofluid collection device, comprising:
a first layer, including a polymer substrate having a surface and a plurality of interconnected microchannels arranged in a repeating hexagonal pattern in the surface;
a second layer, including a hydrophilic gold coating substantially covering the surface and the microchannels; and
a third layer, including a functionalization coating substantially covering the microchannels.

2. The device of claim 1, wherein the functionalization coating is comprised of one of the following: a monothiol thioglycolic acid; sodium 3-mercapto-1-propanesulfonate; a 5mer peptide; and a 7mer peptide.

3. The device of claim 1, wherein the functionalization coating promotes a contact angle between a biofluid and a channel surface that is one of the following: less than 75 degrees; less than 66 degrees; less than 35 degrees; and less than 30 degrees.

4. The device of claim 1, further comprising:
a fluid-impermeable base;
a wicking coupler;
a wicking pump; and
one or more sensors for measuring a characteristic of an analyte in biofluid.

5. The device of claim 4, further comprising:
one or more of the following sensors: a volumetric sweat rate sensor, a micro-thermal flow rate sensor, a GSR sensor, a sweat conductivity sensor, an impedance sensor, a capacitance sensor, and a temperature sensor.

6. A biofluid collection device, comprising:
a polymer substrate; and
a network of interconnected microchannels in a surface of the substrate, the network comprising three or more open channels that repeatedly intersect to form a plurality of fluid transport pathways; wherein said pathways are configured to propagate fluid in a plurality of directions.

7. The device of claim 6, further comprising a hydrophilic gold layer that substantially covers the surface and the network.

8. The device of claim 6, further comprising a functionalization layer that substantially covers the network.

9. The device of claim 6, wherein said network has a wicking volume of one or more of the following: less than one thousand nL/cm², less than five hundred nL/cm², less than one hundred nL/cm².

10. The device of claim 6, wherein said network is configured to have a storage stability duration of one of the following: 30 days; 1 year; and 2 years.

11. The device of claim 6, wherein said network is configured to have a usage stability duration of one of the following: 1 day; 7 days; and 30 days.

12. The device of claim 6, wherein said channels have a height-to-width aspect ratio of one of: 1:2; 1:1; 1:1.5; 2:1; and 3:1.
